(19) United States
(12) Patent Application Publication
(10) Pub. No.: US 2004/0092710 A1
(43) Pub. Date: May 13, 2004

(54) Nucleotide sequences coding for the cdsA gene

(76) Inventors: Madhavan Nampoothiri K., Kerala (IN); Bettina Mockel, Dusseldorf (DE); Walter Pfefferle, Halle (DE); Lothar Eggeling, Julich (DE); Hermann Sahm, Julich (DE)

Correspondence Address: SMITH, GAMBRELL & RUSSELL, LLP, 1850 M STREET, N.W., SUITE 800, WASHINGTON, DC, 20036, US

(21) Appl. No.: 09/853,641

(22) Filed: May 14, 2001

Related U.S. Application Data
(63) Continuation-in-part of application No. 09/577,856, filed on May 25, 2000, now abandoned.

(30) Foreign Application Priority Data
May 4, 2000 (DE) 100 21 828.8

(51) Int. Cl. C07K 1/00; C07K 14/00; C07K 17/00

(52) U.S. Cl. 530/350

(57) ABSTRACT

This invention relates to a genetically modified coryneform bacterium, the cdsA gene of which is amplified, and to an isolated polynucleotide, which codes for phosphatidate cytidylyl transferase from coryneform bacteria and to a method for the fermentative production of L-amino acids with amplification of the cdsA gene in the bacteria and to the use of the polynucleotide as a primer or hybridization probe.

### RELATED APPLICATION DATA

This application is a Continuation-In-Part of co-pending U.S. patent application Ser. No. 09/577,856 filed May 25, 2000, which application claims priority under 35 U.S.C. §119 from German Patent Appln. No. 10021828.8, filed in Germany on May 4, 2000. The above-identified U.S. patent application and German patent application are entirely incorporated herein by reference.

The present invention provides genetically modified coryneform bacteria, nucleotide sequences coding for phosphatidate cytidylyl transferase and method for the fermentative production of amino acids, in particular L-lysine, using coryneform bacteria, in which the cdsA gene, which codes for phosphatidate cytidylyl transferase, is amplified. All references cited herein are expressly incorporated by reference. Incorporation by reference is also designated by the term “I.B.R.” following any citation.

### BACKGROUND ART

Amino acids, in particular L-lysine, are used in human medicine and in the pharmaceuticals industry, but in particular in animal nutrition.

It is known that amino acids are produced by fermentation of strains of coryneform bacteria, in particular Corynebacterium glutamicum. Due to their great significance, efforts are constantly being made to improve the production method. Improvements to the method may relate to measures concerning fermentation technology, for example stirring and oxygen supply, or to the composition of the nutrient media, such as for example sugar concentration during fermentation, or to working up to yield the product by, for example, ion exchange chromatography, or to the intrinsic performance characteristics of the microorganism itself.

The performance characteristics of these microorganisms are improved using methods of mutagenesis, selection and mutant selection. In this manner, strains are obtained which are resistant to antimetabolites, such as for example the lysine analogue S-(2-aminoethyl)cysteine, or are auxotrophic for regulatorily significant amino acids and produce L-amino acids, such as for example L-lysine.

For some years, methods of recombinant DNA technology have moreover been used to improve strains of Corynebacterium which produce amino acids by amplifying individual biosynthesis genes and investigating the effect on amino acid production. Review articles on this subject may be found inter alia in Kinoshita (“Glutamic Acid Bacteria”, in: Biology of Industrial Microorganisms, Demain and Solomon (Eds.) I.B.R., Benjamin Cummings, London, UK, 1985, 115-142) I.B.R., Hilliger (BioTec 2, 40-44 (1991)) I.B.D., Eggeling (Amino Acids 6:261-272 (1994)) I.B.D., Jetten and Sinskey (Critical Reviews in Biotechnology 15, 73-103 (1995)) I.B.R. and Sahm et al. (Annuals of the New York Academy of Science 782, 25-39 (1996)) I.B.D.

### OBJECT OF THE INVENTION

The object of the present invention was to provide novel auxiliaries for the improved fermentative production of amino acids, in particular L-lysine.

This object is achieved by a genetically modified coryneform bacterium, the cdsA gene of which, which codes for phosphatidate cytidylyl transferase, is amplified.

Amino acids, in particular L-lysine, are used in human medicine, in the pharmaceuticals industry and in particular in animal nutrition. There is accordingly general interest in providing novel improved methods for the production of amino acids, in particular L-lysine.

Any subsequent mention of L-lysine or lysine should be taken to mean not only the base, but also salts, such as for example lysine monohydrochloride or lysine sulfate.

### SUMMARY OF THE INVENTION

The new DNA sequence of C. glutamicum which codes for the cdsA gene and which as a constituent of the present invention is SEQ ID NO 1 and related sequences. The amino acid sequence of the corresponding gene product of the cdsA gene has furthermore been derived from the present DNA sequence. The resulting amino acid sequence of the cdsA gene product is SEQ ID NO 2 and related sequences.

### BRIEF DESCRIPTION OF THE DRAWINGS

The present invention will be further understood with reference to the drawing offered here for illustration only and not in limitation of this invention.

FIG. 1: Map of the plasmid pJC1cdsA

FIG. 2: Growth of C. glutamicum ATCC 13032 and ATCC 13032/pJCcdsA at 40° C.

### DETAILED DESCRIPTION OF THE INVENTION

The present invention provides a genetically modified coryneform bacterium, in which the cdsA gene of which, which codes for phosphatidate cytidylyl transferase, is amplified.

In this connection, the term “amplification” describes the increase in the intracellular activity of one or more enzymes in a microorganism, which enzymes are coded by the corresponding DNA.

Amplification may be achieved by means of various manipulations of the bacterial cells.

Amplification, in particular overexpression, may be achieved by increasing the copy number of the corresponding genes, by using a strong promoter or by mutating the promoter and regulation region or the ribosome-binding site located upstream from the structural gene. Expression cassettes incorporated upstream from the structural gene act in the same manner. It is additionally possible to increase expression during fermentative L-lysine production by means of inducible promoters. It is also possible to use a gene which codes for a corresponding enzyme having an elevated activity. Expression is also improved by measures to extend the lifetime of the mRNA. An overall increase in enzyme activity is moreover achieved by preventing degradation of the enzyme. These measures may optionally be combined at will.

The microorganisms, provided by the present invention, may produce L-amino acids, in particular L-lysine, from glucose, sucrose, lactose, fructose, maltose, molasses, starch, cellulose or from glycerol and ethanol. The microorganisms may comprise representatives of the coryneform bacteria in particular of the genus Corynebacterium. Within the genus Corynebacterium, the species Corynebacterium glutamicum may in particular be mentioned, which is known in specialist circles for its ability to produce L-amino acids.

Suitable strains of the genus Corynebacterium, in particular of the species Corynebacterium glutamicum, are for example the known wild type strains.

Corynebacterium glutamicum ATCC13032

Corynebacterium acetoglutamicum ATCC15806

Corynebacterium acetoacidophilum ATCC13870

Corynebacterium thermoaminogenes FERM BP-1539

Corynebacterium melassecola ATCC17965

Brevibacterium flavum ATCC1406

Brevibacterium lactofermentum ATCC13869 and

Brevibacterium divaricatum ATCC14020

and L-lysine producing mutants or strains produced therefrom, such as for example

Corynebacterium glutamicum FERM-P 1709

Brevibacterium flavum FERM-P 1708

Brevibacterium lactofermentum FERM-P 1712

Corynebacterium glutamicum FERM-P 6463

Corynebacterium glutamicum FERM-P 6464 and

Corynebacterium glutamicum DSM5715.

The present invention also provides an isolated polynucleotide from coryneform bacteria containing a polynucleotide sequence selected from the group

a) polynucleotide which is at least 70% homologous to a polynucleotide which codes for a polypeptide containing the amino acid sequence of SEQ ID no. 2,

b) polynucleotide which codes for a polypeptide which contains an amino acid sequence which is at least 70% homologous to the amino acid sequence of SEQ ID no. 2,

c) polynucleotide which is complementary to the polynucleotides of a) or b), and

d) polynucleotide containing at least 15 successive nucleotides of the polynucleotide sequence of a), b) or c).

For the purposes of the present application, a polynucleotide sequence is “homologous” to the sequence according to the invention if the base composition and sequence thereof at least 70%, preferably at least 80%, particularly preferably at least 90% matches the sequence according to the invention. According to the present invention, a “homologous protein” should be taken to mean proteins which have an amino acid sequence which at least 70%, preferably at least 80%, particularly preferably at least 90% matches the amino acid sequence which is coded by the cdsA gene (SEQ ID no. 1), wherein “matching” should be taken to mean that the corresponding amino acids are either identical or comprise mutually homologous amino acids. “Homologous amino acids” are those having corresponding properties, in particular with regard to charge, hydrophobicity, steric properties etc.

The present invention moreover provides a polynucleotide as described above, wherein it preferably comprises replicable DNA containing:

(i) the nucleotide sequence shown in SEQ ID no. 1, or

(ii) at least one sequence which corresponds to the sequence (i) within the degeneration range of the genetic code, or

(iii) at least one sequence which hybridizes with the complementary sequence to sequence (i) or (ii) and optionally

(iv) functionally neutral mutations in (i) which give rise to the same or a homologous amino acid.

The relative degree of substitution or mutation in the polynucleotide or amino acid sequence to produce a desired percentage of sequence identity can be established or determined by well-known methods of sequence analysis. These methods are disclosed and demonstrated in Bishop, et al. “DNA & Protein Sequence Analysis (A Practical Approach”), Oxford Univ. Press, Inc. (1997) I.B.R. and by Steinberg, Michael “Protein Structure Prediction” (A Practical Approach), Oxford Univ. Press, Inc. (1997) I.B.R. Hybridization of complementary sequences can occur at varying degrees of stringency. Sambrook et al.: Molecular Cloning, A Laboratory Manual (Cold Spring Harbor Laboratory Press, 1989) I.B.R.

Hybridization of complementary sequences can occur at varying degrees of stringency. Sambrook et al.: Molecular Cloning, A Laboratory Manual (Cold Spring Harbor Laboratory Press, 1989) I.B.R. Instructions for identifying DNA sequences by means of hybridization can be found by the expert, inter alia, in the handbook “The DIG System Users Guide for Filter Hybridization” from Boehringer Mannheim GmbH (Mannheim, Germany, 1993)I.B.R. and in Liebl et al. (International Journal of Systematic Bacteriology (1991) 41: 255-260) I.B.R.

Comprehensive descriptions can be found in known textbooks of genetics and molecular biology, such as e. g. that by Hagemann (“Allgemeine Genetik” [General Genetics], Gustav Fischer Verlag, Stuttgart, 1986) I.B.R.

Possible mutations are transitions, transversions, insertions and deletions. Depending on the effect of the amino acid exchange on the enzyme activity, missense mutations or nonsense mutations are referred to. Insertions or deletions of at least one base pair in a gene lead to frame shift mutations, as a consequence of which incorrect amino acids are incorporated or translation is interrupted prematurely. Deletions of several codons typically lead to a complete loss of the enzyme activity.

Instructions on generation of such mutations are prior art and can be found in known textbooks of genetics and molecular biology, such as e. g. the textbook by Knippers (“Molekulare Genetik” [Molecular Genetics], 6th edition, Georg Thieme Verlag, Stuttgart, Germany, 1995) I.B.R., that by Winnacker (“Gene und Klone” [Genes and Clones], VCH Verlagsgesellschaft, Weinheim, Germany, 1990) I.B.R. or that by Hagemann (“Allgemeine Genetik” [General Genetics], Gustav Fischer Verlag, Stuttgart, 1986) I.B.R.

The present invention also provides

a preferably recombinant polynucleotide replicable in coryneform bacteria, which polynucleotide comprises the nucleotide sequence SEQ ID no. 1,

a polynucleotide which codes for a polypeptide which comprises the amino acid sequence SEQ ID no. 2

a vector containing the DNA sequence of C. glutamicum which codes for the cdsA gene, contained in the vector pJC1cdsA, deposited in Corynebacterium glutamicum under the number 13252,

and coryneform bacteria acting as host cell which contain the vector or in which the cdsA gene is amplified.

The present invention also provides polynucleotides which contain the complete gene with the polynucleotide sequence according to SEQ ID no. 1 or fragments thereof and which are obtainable by screening by means of hybridization of a suitable gene library with a probe which contains the sequence of the stated polynucleotide according to SEQ ID no. 1 or a fragment thereof and isolation of the stated DNA sequence.

Polynucleotide sequences according to the invention are also suitable as hybridization probes for RNA, cDNA and DNA in order to isolate full length cDNA which code for phosphatidate cytidylyl transferase and to isolate such cDNA or genes, which exhibit a high level of similarity with the sequence of phosphatidate cytidylyl transferase.

Polynucleotide sequences according to the invention are furthermore suitable as primers for the polymerase chain reaction (PCR) for the production of DNA which codes for phosphatidate cytidylyl transferase.

Such oligonucleotides acting as probes or primers may contain more than 30, preferably up to 30, particularly preferably up to 20, very particularly preferably at least 15 successive nucleotides. Oligonucleotides having a length of at least 40 or 50 nucleotides are also suitable.

“Isolated” means separated from its natural environment.

“Polynucleotide” generally relates to polyribonucleotides and polydeoxyribonucleotides, wherein the RNA or DNA may be unmodified or modified.

“Polypeptides” are taken to mean peptides or proteins which contain two or more amino acids connected by peptide bonds.

The polypeptides according to the invention include a polypeptide according to SEQ ID no. 2, in particular those having the biological activity of phosphatidate cytidylyl transferase and also those which are at least 70%, preferably at least 80%, homologous to the polypeptide according to SEQ ID no. 2 and in particular which exhibit 90% to 95% homology to the polypeptide according to SEQ ID no. 2 and exhibit the stated activity.

The invention moreover relates to a method for the fermentative production of amino acids, in particular L-lysine, using coryneform bacteria, which in particular already produce an amino acid and in which the nucleotide sequences which code for the cdsA gene are amplified, in particular overexpressed.

The present invention presents for the first time the cdsA gene of C. glutamicum which codes for phosphatidate cytidylyl transferase.

The cdsA gene or also other genes from C. glutamicum are isolated by initially constructing a gene library of this microorganism in E. coli. The construction of gene libraries is described in generally known textbooks and manuals. Examples which may be mentioned are the textbook by Winnacker: Gene und Klone, Eine Einfuthrung in die Gentechnologie (Verlag Chemie, Weinheim, Germany, 1990) I.B.R. or the manual by Sambrook et al.: Molecular Cloning, A Laboratory Manual (Cold Spring Harbor Laboratory Press, 1989) I.B.R. One very well known gene library is that of E. coli K-12 strain W3110, which was constructed by Kohara et al. (Cell 50, 495-508 (1987)) I.B.R. in λ-vectors. Bathe et al. (Molecular and General Genetics, 252:255-265, 1996) I.B.R. describe a gene library of C. glutamicum ATCC13032, which was constructed using the cosmid vector SuperCos I (Wahl et al., 1987, Proceedings of the National Academy of Sciences USA, 84:2160-2164 I.B.R.) in E. coli K-12 strain NM554 (Raleigh et al., 1988, Nucleic Acids Research 16:1563-1575 I.B.R.). Börmann et al. (Molecular Microbiology 6(3), 317-326, 1992)) I.B.R. also describe a gene library of C. glutamicum ATCC13032, using cosmid pHC79 (Hohn and Collins, Gene 11, 291-298 (1980)) I.B.R. A gene library of C. glutamicum in E. coli may also be produced using plasmids such as pBR322 (Bolivar, Life Sciences, 25, 807-818 (1979) I.B.R.) or pUC9 (Vieira et al., 1982, Gene, 19:259-268 I.B.R.). Suitable hosts are in particular those E. coli strains with restriction and recombination defects. One example of such a strain is the strain DH5αmcr, which has been described by Grant et al. (Proceedings of the National Academy of Sciences USA, 87 (1990) 4645-4649) I.B.R. The long DNA fragments cloned with the assistance of cosmids may then in turn be sub-cloned in usual vectors suitable for sequencing and then be sequenced, as described, for example, in Sanger et al. (Proceedings of the National Academy of Sciences of the United States of America, 74:5463-5467, 1977) I.B.R.

The novel DNA sequence from C. glutamicum which codes for the cdsA gene and, as SEQ ID no. 1, is provided by the present invention, was obtained in this manner. The amino acid sequence of the corresponding protein was furthermore deduced from the above DNA sequence using the methods described above. SEQ ID no. 2 shows the resultant amino acid sequence of the product of the cdsA gene.

Coding DNA sequences arising from SEQ ID no. 1 due to the degeneracy of the genetic code are also provided by the present invention. DNA sequences which hybridize with SEQ ID no. 1 or parts of SEQ ID no. 1 are similarly provided by the invention. Conservative substitutions of amino acids in proteins, for example the substitution of glycine for alanine or of aspartic acid for glutamic acid, are known in specialist circles as “sense mutations”, which result in no fundamental change in activity of the protein, i.e. they are functionally neutral. It is furthermore known that changes to the N and/or C terminus of a protein do not substantially impair or may even stabilize the function thereof. The person skilled in the art will find information in this connection inter alia in Ben-Bassat et al. (Journal of Bacteriology 169:751-757 (1987)) I.B.R., in O'Regan et al. (Gene 77:237-251 (1989)) I.B.R., in Sahin-Toth et al. (Protein Sciences 3:240-247 (1994)) I.B.R., in Hochuli et al. (Bio/Technology 6:1321-1325 (1988)) I.B.R. and in known textbooks of genetics and molecular biology. Amino acid sequences arising in a corresponding manner from SEQ ID no. 2 are also provided by the present invention.

DNA sequences which hybridize with SEQ ID no. 1 or parts of SEQ ID no. 1 are similarly provided by the invention. Finally, DNA sequences produced by the polymerase chain reaction (PCR) using oligonucleotide primers obtained from SEQ ID no. 1 are also provided by the present invention. Such oligonucleotides typically have a length of at least 15 nucleotides.

The person skilled in the art may find instructions for identifying DNA sequences by means of hybridization inter alia in the manual “The DIG System Users Guide for Filter Hybridization” from Boehringer Mannheim GmbH (Mannheim, Germany, 1993) I.B.R. and in Liebl et al. (International Journal of Systematic Bacteriology (1991) 41: 255-260) I.B.R. The person skilled in the art may find instructions for amplifying DNA sequences using the polymerase chain reaction (PCR) inter alia in the manual by Gait: Oligonucleotide synthesis: a practical approach (IRL Press, Oxford, UK, 1984) I.B.R. and in Newton & Graham, PCR (Spektrum Akademischer Verlag, Heidelberg, Germany, 1994) I.B.R.

During work on the present invention, it proved possible to establish that coryneform bacteria produce amino acids, in particular L-lysine, in an improved manner once the cdsA gene has been amplified.

The genes or gene constructs under consideration may either be present in plasmids in a variable copy number or be integrated into the chromosome and amplified. Alternatively, overexpression of the genes concerned may also be achieved by modifying the composition of the media and culture conditions.

The person skilled in the art will find guidance in this connection inter alia in Martin et al. (Bio/Technology 5, 137-146 (1987)) I.B.R., in Guerrero et al. (Gene 138, 35-41 (1994)) I.B.R., Tsuchiya and Morinaga (Bio/Technology 6, 428-430 (1988)) I.B.R., in Eikmanns et al. (Gene 102, 93-98 (1991)) I.B.R., in European patent EPS 0 472 869 I.B.R., in U.S. Pat. No. 4,601,893 I.B.R., in Schwarzer and Puhler (Bio/Technology 9, 84-87 (1991) I.B.R., in Reinscheid et al. (Applied and Environmental Microbiology 60, 126-132 (1994)) I.B.R., in LaBarre et al. (Journal of Bacteriology 175, 1001-1007 (1993)) I.B.R., in patent application WO 96/15246 I.B.R., in Malumbres et al. (Gene 134, 15-24 (1993)) I.B.R., in Japanese published patent application JP-A-10-229891 I.B.R., in Jensen and Hammer (Biotechnology and Bioengineering 58, 191-195 (1998)) I.B.R., in Makrides (Microbiological Reviews 60:512-538 (1996)) I.B.R. and in known textbooks of genetics and molecular biology.

By way of example, the cdsA gene according to the invention was overexpressed with the assistance of plasmids.

Suitable plasmids are those which are replicated and expressed in coryneform bacteria. Numerous known plasmid vectors, such as for example pZ1 (Menkel et al., Applied and Environmental Microbiology (1989) 64: 549-554), pEKEx1 (Eikmanns et al., Gene 102:93-98 (1991)) or pHS2-1 (Sonnen et al., Gene 107:69-74 (1991)) are based on the cryptic plasmids pHM1519, pBL1 or pGA1. Other plasmid vectors, such as for example those based on pCG4 (U.S. Pat. No. 4,489,160), or pNG2 (Serwold-Davis et al., FEMS Microbiology Letters 66, 119-124 (1990)), or pAG1 (U.S. Pat. No. 5,158,891) may be used in the same manner.

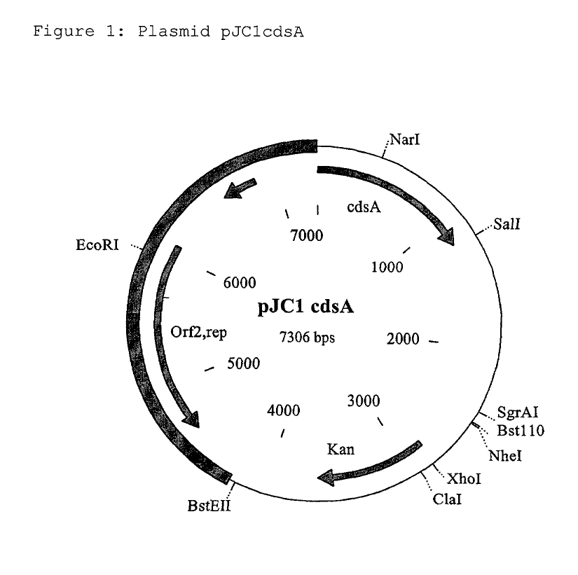

One example of a plasmid by means of which the cdsA gene may be overexpressed is pJC1cdsA (FIG. 1), which is based on the E. coli-C. glutamicum shuttle vector pJC1 (Cremer et al., 1990, Molecular and General Genetics 220: 478-480 I.B.R.) and contains the DNA sequence of C. glutamicum which codes for the cdsA gene. It is contained in the strain DSM5715/pJC1cdsA.

Further suitable plasmid vectors are those with the assistance of which gene amplification may be performed by integration into the chromosome, as has for example been described by Reinscheid et al. (Applied and Environmental Microbiology 60, 126-132 (1994)) I.B.R. for the duplication or amplification of the hom-thrB operon. In this method, the complete gene is cloned into a plasmid vector which can replicate in a host (typically E. coli), but not in C. glutamicum. Vectors which may be considered are, for example, pSUP301 (Simon et al., Bio/Technology 1, 784-791 (1983)) I.B.R., pK18mob or pK19mob (Schafer et al., Gene 145, 69-73 (1994) I.B.R.), pGEM-T (Promega corporation, Madison, Wis., USA), pCR2.1-TOPO (Shuman (1994). Journal of Biological Chemistry 269:32678-84; U.S. Pat. No. 5,487,993 I.B.R.), pCR®Blunt (Invitrogen, Groningen, Netherlands; Bernard et al., Journal of Molecular Biology, 234: 534-541 (1993) I.B.R.) or pEM1 (Schrumpf et al, 1991, Journal of Bacteriology 173:4510-4516 I.B.R.). The plasmid vector which contains the gene to be amplified is then transferred into the desired strain of C. glutamicum by conjugation or transformation. The conjugation method is described, for example, in Schäfer et al. (Applied and Environmental Microbiology 60, 756-759 (1994)) I.B.R. Transformation methods are described, for example, in Thierbach et al. (Applied Microbiology and Biotechnology 29, 356-362 (1988)) I.B.R., Dunican and Shivnan (Bio/Technology 7, 1067-1070 (1989)) I.B.R. and Tauch et al. (FEMS Microbiological Letters 123, 343-347 (1994)) I.B.R. After homologous recombination by means of “crossing over”, the resultant strain contains at least two copies of the gene in question.

It may additionally be advantageous for the production of amino acids, in particular L-lysine, to amplify or overexpress not only the cdsA gene, but also one or more enzymes of the particular biosynthetic pathway, of glycolysis, of anaplerotic metabolism, of the citric acid cycle or of amino acid export.

For the production of L-lysine, for example, it is thus possible simultaneously to amplify, in particular overexpress or amplify, one or more genes selected from the group

the dapA gene which codes for dihydropicolinate synthase (EP-B 0 197 335 I.B.R.), or

the dapE gene which codes for succinyldiaminopimelate desuccinylase, or

the lysC gene which codes for a feed back resistant aspartate kinase (Kalinowski et al. (1990), Molecular and General Genetics 224, 317-324 I.B.R.), or

the gap gene which codes for glyceraldehyde-3-phosphate dehydrogenase (Eikmanns (1992), Journal of Bacteriology 174:6076-6086 I.B.R.), or

the tpi gene which codes for triosephosphate isomerase (Eikmanns (1992), Journal of Bacteriology 174:6076-6086 I.B.R.), or

the pgk gene which codes for 3-phosphoglycerate kinase (Eikmanns (1992), Journal of Bacteriology 174:6076-6086 I.B.R.), or

the pyc gene which codes for pyruvate carboxylase (DE-A-19831609 I.B.R.), or

the mqo gene which codes for malate:quinone oxidoreductase (Molenaar et al., European Journal of Biochemistry 254, 395-403 (1998) I.B.R.), or

the lysE gene which codes for lysine export (DE-A-195 48 222 I.B.R.)

It may furthermore be advantageous for the production of amino acids, in particular L-lysine, in addition to amplifying the cdsA gene, simultaneously to attenuate

the pck gene which codes for phosphoenolpyruvate carboxykinase (DE 199 50 409.1 I.B.R., DSM 13047 I.B.R.) and/or

the pgi gene which codes for glucose 6-phosphate isomerase (U.S. Ser. No. 09/396,478 I.B.R., DSM 12969 I.B.R.) and/or

the poxB gene which codes for pyruvate oxidase (DE:1995 1975.7 I.B.R.).

It may furthermore be advantageous for the production of amino acids, in particular L-lysine, in addition to overexpressing the cdsA gene, to suppress unwanted secondary reactions (Nakayama: “Breeding of Amino Acid Producing Microorganisms”, in: Overproduction of Microbial Products, Krumphanzl, Sikyta, Vanek (eds.), Academic Press, London, UK, 1982 I.B.R.).

For the purposes of amino acid production, in particular of L-lysine, the microorganisms produced according to the invention may be cultured continuously or discontinuously using the batch process or the fed batch process or repeated fed batch process. A summary of known culture methods is given in the textbook by Chmiel (Bioprozesstechnik 1. Einführung in die Bioverfahrenstechnik (Gustav Fischer Verlag, Stuttgart, 1991) I.B.R.) or in the textbook by Storhas (Bioreaktoren und periphere Einrichtungen (Vieweg Verlag, Braunschweig/ Wiesbaden, 1994) I.B.R.).

The culture medium to be used must adequately satisfy the requirements of the particular strains. Culture media for various microorganisms are described in “Manual of Methods for General Bacteriology” from the American Society for Bacteriology (Washington D.C., USA, 1981) I.B.R. Carbon sources which may be used are sugars and carbohydrates, such as glucose, sucrose, lactose, fructose, maltose, molasses, starch and cellulose for example, oils and fats, such as soya oil, sunflower oil, peanut oil and coconut oil for example, fatty acids, such as palmitic acid, stearic acid and linoleic acid for example, alcohols, such as glycerol and ethanol for example, and organic acids, such as acetic acid for example. These substances may be used individually or as a mixture. Nitrogen sources which may be used comprise organic compounds containing nitrogen, such as peptones, yeast extract, meat extract, malt extract, corn steep liquor, soya flour and urea or inorganic compounds, such as ammonium sulfate, ammonium chloride, ammonium phosphate, ammonium carbonate and ammonium nitrate. The nitrogen sources may be used individually or as a mixture. Phosphorus sources which may be used are phosphoric acid, potassium dihydrogen phosphate or dipotassium hydrogen phosphate or the corresponding salts containing sodium. The culture medium must furthermore contain metal salts, such as for example magnesium sulfate or iron sulfate, which are necessary for growth. Finally, essential growth-promoting substances such as amino acids and vitamins may also be used in addition to the above-stated substances. Suitable precursors may furthermore be added to the culture medium. The stated feed substances may be added to the culture as a single batch or be fed appropriately during culturing.

Basic compounds, such as sodium hydroxide, potassium hydroxide, ammonia or ammonia water, or acidic compounds, such as phosphoric acid or sulfuric acid, are used appropriately to control the pH of the culture. Foaming may be controlled by using antifoaming agents such as fatty acid polyglycol esters for example. Plasmid stability may be maintained by the addition to the medium of suitable selectively acting substances, for example antibiotics. Oxygen or oxygen-containing gas mixtures, such as air for example, are introduced into the culture in order to maintain aerobic conditions. The temperature of the culture is normally from 20° C. to 45° C. and preferably from 25° C. to 40° C. The culture is continued until the maximum quantity of lysine has formed. This objective is normally achieved within 10 hours to 160 hours.

Analysis of L-lysine may be performed by anion exchange chromatography with subsequent ninhydrin derivatization, as described in Spackman et al. (Analytical Chemistry, 30, (1958), 1190) I.B.R.

The following microorganism has been deposited with Deutsche Sammlung für Mikrorganismen und Zellkulturen (DSMZ, Braunschweig, Germany) in accordance with the Budapest Treaty:

Corynebacterium glutamicum strain DSM5715/pJC1cdsA as DSM 13252

The purpose of the method according to the invention is the fermentative production of amino acids, in particular L-lysine.

Key to the Figures

FIG. 1: Map of the plasmid pJC1cdsA

The abbreviations and names are defined as follows.

Orf2, rep: plasmid-coded replication origin, C. glutamicum (from pHM1519)

cdsA: cdsA (phosphatidate cytidylyl transferase) gene from C. glutamicum ATCC13032

Kan: kanamycin resistance gene

NarI restriction site of the restriction enzyme NarI

SalI: restriction site of the restriction enzyme SalI

SgrAI restriction site of the restriction enzyme SgrAI

Bst1107 Restriction site of the restriction enzyme Bst1107

NheI restriction site of the restriction enzyme NheI

XhoI restriction site of the restriction enzyme XhoI

ClaI restriction site of the restriction enzyme ClaI

BstEII restriction site of the restriction enzyme BstEII

EcoRI restriction site of the restriction enzyme EcoRI

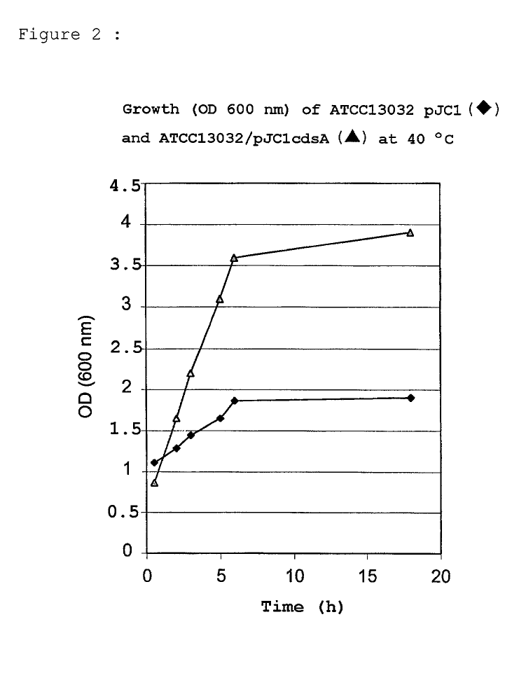

FIG. 2:

Growth of C. glutamicum ATCC 13032 and ATCC 13032/pJCcdsA at 40° C.

OD: optical density

### EXAMPLES

The present invention is illustrated in greater detail by the following practical examples.

### Example 1

Production of a genomic cosmid gene library from Corynebacterium glutamicum ATCC 13032

Chromosomal DNA from Corynebacterium glutamicum ATCC 13032 was isolated as described in Tauch et al., (1995, Plasmid 33:168-179) I.B.R. and partially cleaved with the restriction enzyme Sau3AI (Amersham Pharmacia, Freiburg, Germany, product description Sau3AI, code no. 27-0913-02). The DNA fragments were dephosphorylated with shrimp alkaline phosphatase (Roche Molecular Biochemicals, Mannheim, Germany, product description SAP, code no. 1758250). The DNA of cosmid vector SuperCos1 (Wahl et al. (1987) Proceedings of the National Academy of Sciences USA 84:2160-2164) I.B.R., purchased from Stratagene (La Jolla, USA, product description SuperCos1 Cosmid Vector Kit, code no. 251301) was cleaved with the restriction enzyme XbaI (Amersham Pharmacia, Freiburg, Germany, product description XbaI, Code no. 27-0948-02) and also dephosphorylated with shrimp alkaline phosphatase. The cosmid DNA was then cleaved with the restriction enzyme BamHI (Amersham Pharmacia, Freiburg, Germany, product description BamHI, code no. 27-0868-04). Cosmid DNA treated in this manner was mixed with the treated ATCC 13032 DNA and the batch was treated with T4 DNA ligase (Amersham Pharmacia, Freiburg, Germany, product description T4 DNA Ligase, code no. 27-0870-04). The ligation mixture was then packed in phages using Gigapack II XL Packing Extracts (Stratagene, La Jolla, USA, product description Gigapack II XL Packing Extract, code no. 200217). E. coli strain NM554 (Raleigh et al. 1988, Nucleic Acid Research 16:1563-1575 I.B.R.) was infected by suspending the cells in 10 mM MgSO₄ and mixing them with an aliquot of the phage suspension. The cosmid library was infected and titred as described in Sambrook et al. (1989, Molecular Cloning: A laboratory Manual, Cold Spring Harbor I.B.R.), the cells being plated out on LB agar (Lennox, 1955, Virology, 1:190 I.B.R.) with 100 mg/l of ampicillin. After overnight incubation at 37° C., individual recombinant clones were selected.

### Example 2

Isolation and Sequencing of the cdsA Gene.

Cosmid DNA from an individual colony was isolated in accordance with the manufacturer's instructions using the Qiaprep Spin Miniprep Kit (Product No. 27106, Qiagen, Hilden, Germany) and partially cleaved with the restriction enzyme Sau3AI (Amersham Pharmacia, Freiburg, Germany, product description Sau3AI, code no. 27-0913-02). The DNA fragments were dephosphorylated with shrimp alkaline phosphatase (Roche Molecular Biochemicals, Mannheim, Germany, product description SAP, code no. 1758250). Once separated by gel electrophoresis, the cosmid fragments of a size of 1500 to 2000 bp were isolated using the QiaExII Gel Extraction Kit (product no. 20021, Qiagen, Hilden, Germany). The DNA of the sequencing vector pZero-1 purchased from Invitrogen (Groningen, Netherlands, product description Zero Background Cloning Kit, product no. K2500-01) was cleaved with the restriction enzyme BamHI (Amersham Pharmacia, Freiburg, Germany, product description BamHI, Product No. 27-0868-04). Ligation of the cosmid fragments into the sequencing vector pZero-1 was performed as described by Sambrook et al. (1989, Molecular Cloning: A laboratory Manual, Cold Spring Harbor) I.B.R., the DNA mixture being incubated overnight with T4 ligase (Pharmacia Biotech, Freiburg, Germany). This ligation mixture was then electroporated into the E. coli strain DH5AMCR (Grant, 1990, Proceedings of the National Academy of Sciences U.S.A., 87:4645-4649 I.B.R.) (Tauch et al. 1994, FEMS Microbiol Letters, 123:343-7 I.B.R.) and plated out onto LB agar (Lennox, 1955, Virology, 1:190 I.B.R.) with 50 mg/l of Zeocin. Plasmids of the recombinant clones were prepared using the Biorobot 9600 (product no. 900200, Qiagen, Hilden, Germany). Sequencing was performed using the dideoxy chain termination method according to Sanger et al. (1977, Proceedings of the National Academy of Sciences U.S.A., 74:5463-5467 I.B.R.) as modified by Zimmermann et al. (1990, Nucleic Acids Research, 18:1067) I.B.R. The “RR dRhodamin Terminator Cycle Sequencing Kit” from PE Applied Biosystems (product no. 403044, Weiterstadt, Germany) was used. Separation by gel electrophoresis and analysis of the sequencing reaction was performed in a “Rotiphorese NF” acrylamide/bisacrylamide gel (29:1) (product no. A124.1, Roth, Karlsruhe, Germany) using the “ABI Prism 377” sequencer from PE Applied Biosystems (Weiterstadt, Germany).

The resultant raw sequence data were then processed using the Staden software package (1986, Nucleic Acids Research, 14:217-231 I.B.R.), version 97-0. The individual sequences of the pZero 1 derivatives were assembled into a cohesive contig. Computer-aided coding range analysis was performed using XNIP software (Staden, 1986, Nucleic Acids Research, 14:217-231 I.B.R.). Further analysis was performed using the “BLAST search programs” (Altschul et al., 1997, Nucleic Acids Research, 25:3389-3402 I.B.R.), against the non-redundant database of the “National Center for Biotechnology Information” (NCBI, Bethesda, Md., USA).

The resultant nucleotide sequence is stated in SEQ ID no. 1. Analysis of the nucleotide sequence revealed an open reading frame of 891 base pairs, which was designated the cdsA gene. The cdsA gene codes for a protein of 297 amino acids.

### Example 3

Cloning of the cdsA Gene into Vector pJC1

Chromosomal DNA from Corynebacterium glutamicum ATCC 13032 was isolated as described in Tauch et al., (1995, Plasmid 33:168-179). A DNA fragment bearing the cdsA gene was amplified with the assistance of the polymerase chain reaction. The following primers were used for this purpose:

5′-CGC GGA TCC GTG GCC CAA GCT TTA CGA CGG ATA C-

3′

5′-CGC GGA TCC GGC TCG CAA GGA AAA GGA ACT GAT-3′

Both oligonucleotides bear the sequence for the cleavage site of the restriction enzyme BamHI (underlined nucleotides). The stated primers were synthesized by the company MWG Biotech (Ebersberg, Germany) and the PCR reaction was thus performed in accordance with the standard PCR method of Innis et al., (PCR protocol. A guide to methods and applications, 1990, Academic Press I.B.R.). The primers allow the 1095 bp DNA fragment which bears the cdsA gene from Corynebacterium glutamicum to be amplified.

Once separated by gel electrophoresis, the PCR fragment was isolated from the agarose gel using the QiaExII Gel Extraction Kit (product no. 20021, Qiagen, Hilden, Germany).

The PCR fragment obtained in this manner was completely cleaved with the restriction enzyme BamHI. The 1087 bp cdsA fragment was isolated from the agarose gel using the QiaExII Gel Extraction Kit (product no. 1087, Qiagen, Hilden, Germany).

The vector used was the E. coli-C. glutamicum shuttle vector pJC1 (Cremer et al., 1990, Molecular and General Genetics 220: 478-480 I.B.R.). This plasmid was also completely cleaved with the restriction enzyme BamHI and then dephosphorylated with shrimp alkaline phosphatase (Roche Diagnostics GmbH, Mannheim, Germany, product description SAP, product no. 1758250).

The cdsA fragment obtained in this manner was mixed with the prepared pJC1 vector and the batch was treated with T4 DNA ligase (Amersham Pharmacia, Freiburg, Germany, product description T4 DNA Ligase, code no. 27-0870-04). The ligation batch was then transformed into E. coli strain DH5α (Hanahan, in: DNA cloning. A practical approach. Vol. I. IRL-Press, Oxford, Washington DC, USA I.B.R.). Plasmid-bearing cells were selected by plating the transformation batch out onto LB agar (Lennox, 1995, Virology, 1:190 I.B.R.) with 50 mg/l of kanamycin. After overnight incubation at 37° C., individual recombinant clones were selected. Plasmid DNA was isolated from a transformant in accordance with the manufacturer's instructions using the Qiaprep Spin Miniprep Kit (product no. 27106, Qiagen, Hilden, Germany) and cleaved with the restriction enzyme BamHI in order to check the plasmid by subsequent agarose gel electrophoresis. The resultant plasmid was named pJC1cdsA.

### Example 4

Transformation of Strain DSM5715 with Plasmid pJC1cdsA

Strain DSM5715 was then transformed with plasmid pJC1cdsA using the electroporation method described by Liebl et al. (FEMS Microbiology Letters, 53:299-303 (1989)). Transformant selection proceeded on LBHIS agar consisting of 18.5 g/l of brain-heart infusion bouillon, 0.5 M sorbitol, 5 g/l of Bacto tryptone, 2.5 g/l of Bacto yeast extract, 5 g/l of NaCl and 18 g/l of Bacto agar, which had been supplemented with 25 mg/l of kanamycin. Incubation was performed for 2 days at 33° C.

Plasmid DNA was isolated from a transformant using the conventional methods (Peters-Wendisch et al., 1998, Microbiology, 144, 915-927 I.B.R.), cut with the restriction endonuclease BamHI, in order to check the plasmid by subsequent agarose gel electrophoresis. The resultant strain was named DSM5717/pJC1cdsA and deposited with Deutsche Sammlung für Mikrorganismen und Zellkulturen (DSMZ, Braunschweig, Germany) in accordance with the Budapest Treaty as DSM 13252.

### Example 5

Production of Lysine

The C. glutamicum strain DSM5715/pJC1cdsA obtained in Example 5 was cultured in a nutrient medium suitable for the production of L-lysine and the L-lysine content of the culture supernatant was determined.

To this end, the strain was initially incubated for 24 hours at 33° C. on an agar plate with the appropriate antibiotic (brain/heart agar with kanamycin (50 mg/l)). Starting from this agar plate culture, a preculture was inoculated (10 ml of medium in a 100 ml Erlenmeyer flask). The complete medium CgIII was used as the medium for this preculture.

Medium Cg III

| | | |
|---|---|---|
| NaCl | 2.5 | g/l |
| Bacto peptone | 10 | g/l |
| Bacto yeast extract | 10 | g/l |
| Glucose (separately autoclaved) | 2% | (w/v) |

The pH value was adjusted to pH 7.4.

Kanamycin (25 mg/l) was added to this medium. The preculture was incubated for 16 hours at 33° C. on a shaker at 240 rpm. A main culture was inoculated from this preculture, such that the initial OD (660 nm) of the main culture was 0.1. Medium MM was used for the main culture.

Medium MM

| | | |
|---|---|---|
| CSL (Corn Steep Liquor) | 5 | g/l |
| MOPS (morpholinopropanesulfonic | 20 | g/l |
| acid) | | |
| Glucose (separately autoclaved) | 50 | g/l |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| L-Leucine | 0.1 | g/l |
| CaCO₃ | 25 | g/l |

CSL, MOPS and the salt solution were adjusted to pH 7 with ammonia water and autoclaved. The sterile substrate and vitamin solutions, together with the dry-autoclaved CaCO₃ are then added.

Culturing was performed in a volume of 10 ml in a 100 ml Erlenmeyer flask with flow spoilers. Kanamycin (25 mg/l) was added. Culturing was performed at 33° C. and 80% atmospheric humidity.

After 48 hours, the OD was determined at a measurement wavelength of 660 nm using a Biomek 1000 (Beckmann Instruments GmbH, Munich). The quantity of lysine formed was determined using an amino acid analyzer from Eppendorf-BioTronik (Hamburg, Germany) by ion exchange chromatography and post-column derivatization with ninhydrin detection.

Table 1 shows the result of the test.

TABLE 1

| | | Lysine HCl |
|---|---|---|
| Strain | OD(660) | g/L |
| DSM5715/pJC1cdsA | 12.3 | 14.4 |
| DSM5715 | 11.9 | 14.0 |

### Example 6

Improvement of Growth Characteristics

The plasmid pJCcdsA obtained in Example 3 was used to transform C. glutamicum strain ATCC 13032. This strain was transformed as described in Example 4 and checked as in Example 4 by restriction digestion and agarose gel electrophoresis. The strain resultant ATCC 13032/pJCcdsA was cultured in a nutrient medium suitable for determining growth and growth was determined at various temperatures.

To this end, the strain was initially incubated for 24 hours at 30° C. as described in Example 5 on an agar plate with the appropriate antibiotic (brain/heart agar with kanamycin (5 mg/l)). Starting from this agar plate culture, a preculture was inoculated (10 ml of medium in a 100 ml Erlenmeyer flask). The complete medium CgIII stated in Example 5 was used as the medium for this preculture. Kanamycin (25 mg/l) was added to this medium. The preculture was incubated for 16 hours at 30° C. on a shaker at 240 rpm. A main culture was inoculated from this preculture, such that the initial OD (600 nm) of the main culture was 0.7. Medium MM was used for the main culture.

Medium MM

| | | |
|---|---|---|
| MOPS (morpholinopropanesulfonic | 42 | g/l |
| acid) | | |
| Glucose (separately autoclaved) | 40 | g/l |
| (NH₄)₂SO₄ | 20 | g/l |
| KH₂PO₄ | 1.0 | g/l |
| K₂HPO₄ | 1.0 | g/l |
| MgSO₄ * 7 H₂O | 0.25 | g/l |
| CaCl₂ * 2 H₂O | 10 | mg/l |
| FeSO₄ * 7 H₂O | 10 | mg/l |
| MnSO₄ * H₂O | 10 | mg/l |
| ZnSO₄ * H₂O | 1 | mg/l |
| CuSO₄ | 0.2 | mg/l |
| NiCl₂ * 6 H₂O | 0.02 | mg/l |
| Biotin (sterile-filtered) | 0.2 | mg/l |
| Protocatechuic acid | 30 | mg/l |
| (sterile-filtered) | | |

MOPS and the salt solution were adjusted to pH 7 with ammonia water and autoclaved. The sterile substrate and vitamin solutions were then added.

Culturing was performed in a volume of 60 ml in a 500 ml Erlenmeyer flask with flow spoilers. Kanamycin (25 mg/l) was added. Culturing was performed at 40° C. OD was determined at a measurement wavelength of 600 nm using an Ultrospec 3000 (Pharmacia Biotech, Uppsala, Sweden). Table 2 shows the result of the test.

What is claimed is:

1. A genetically modified coryneform bacterium, wherein the cdsA gene thereof, which codes for phosphatidate cytidylyl transferase, is amplified.

2. The genetically modified coryneform bacterium as claimed in claim 1, wherein the starting bacterium (wild type) is selected from the group Corynebacterium glutamicum (ATCC13032), Corynebacterium acetoglutamicum (ATCC15806), Corynebacterium acetoacidophilum (ATCC13870), Corynebacterium thermoaminogenes (FERM BP-1539), Corynebacterium melassecola (ATCC17965), Brevibacterium flavum (ATCC14067), Brevibacterium lactofermentum (ATCC13869) and Brevibacterium divaricatum (ATCC14020), or is selected from the group Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464 and Corynebacterium glutamicum DSM5715.

3. The genetically modified coryneform bacterium as claimed in claim 1, wherein the cdsA gene is amplified by overexpressing the gene.

4. The genetically modified coryneform bacterium as claimed in claim 3, wherein the cdsA gene is over-expressed by increasing the copy number of the gene, by selecting a strong promoter or a regulation region upstream from the reading frame, by mutating the promoter, the regulation region or the ribosome-binding site, by incorporating a suitable expression cassette upstream from the structural gene or by incorporating inducible promoters, by extending the lifetime of the corresponding mRNA, by reducing degradation of the expressed proteins, or by combining two or more of these possibilities.

5. The genetically modified coryneform bacterium as claimed in claim 1, wherein the strain is transformed with a plasmid vector and the plasmid vector bears the nucleotide sequence which codes for the cdsA gene.

6. The genetically modified coryneform bacterium as claimed in claim 1, wherein said bacterium corresponds genotypically to the strain Corynebacterium glutamicum DSM 13252.

7. An isolated polynucleotide from coryneform bacteria containing a polynucleotide sequence selected from the group consisting of:
a) a polynucleotide which is at least 70% homologous to a polynucleotide which codes for a polypeptide which comprises or consists of the amino acid sequence of SEQ ID no. 2,
b) a polynucleotide which codes for a polypeptide which comprises an amino acid sequence which is at least 70% homologous to the amino acid sequence of SEQ ID no. 2,
c) a polynucleotide which is complementary to the polynucleotides of a) or b) and
d) a polynucleotide containing at least 15 successive nucleotides of the polynucleotide sequence of a), b) or c).

8. The polynucleotide as claimed in claim 7, wherein the polynucleotide is a recombinant DNA replicable in coryneform bacteria.

9. The polynucleotide as claimed in claim 7, wherein the polynucleotide is an RNA.

10. The polynucleotide as claimed in claim 7, wherein the DNA, which is capable of replication, comprises:
(i) the nucleotide sequence shown in SEQ ID no. 1, or
(ii) at least one sequence which corresponds to the sequence (i) within the degeneration range of the genetic code, or
(iii) at least one sequence which hybridizes with the complementary sequence to sequence (i) or (ii) and optionally
(iv) functionally neutral mutations in (i) which give rise to homologous amino acids.

11. The polynucleotide sequence as claimed in claim 10, which codes for a polypeptide which comprises the amino acid sequence SEQ ID no. 2.

12. A method for the fermentative production of L-amino acids, wherein, the following step is performed:
a) fermenting L-amino acid producing coryneform bacteria in which at least the cdsA gene or nucleotide sequences coding therefor is/are amplified.

13. The method according to claim 12; wherein the cdsA gene or nucleotide sequences which code for it is amplified by being over-expressed.

14. The method according to claim 12, further comprising:
b) accumulating the L-amino acid in the medium or in the cells of the bacteria.

15. The method according to claim 14, further comprising:
c) isolating the L-amino acid.

16. The method as claimed in claim 12, wherein a genetically modified coryneform bacterium, wherein the cdsA gene, which codes for cyclopropane-mycolic acid synthase, is amplified, is employed.

17. The method as claimed in claim 12, wherein, additional genes, which code for a protein of the biosynthetic pathway of the desired L-amino acid, are amplified in the bacteria.

18. The method as claimed in claim 12, wherein, metabolic pathways which reduce the formation of the desired amino acid are at least partially suppressed in the bacteria.

19. The method as claimed in claim 12, wherein, the amino acid produced is L-lysine.

20. The method as claimed in claim 12, wherein, bacteria are fermented for the production of lysine in which simultaneously one or more of the genes selected from the group consisting of:
a) the dapA gene which codes for dihydropicolinate synthase,
b) the dapE gene which codes for succinyldiamino pimelate desuccinylase,
c) the lysC gene which codes for a feed back resistant aspartate kinase,
d) the tpi gene which codes for triosephosphate isomerase,
e) the gap gene which codes for glyceraldehyde 3-phosphate dehydrogenase,
f) the pgk gene which codes for 3-phosphoglycerate kinase,
g) the pyc gene which codes for pyruvate carboxylase,
h) the mqo gene which codes for malate:quinone oxidoreductase, and
i) the lysE gene which codes for lysine export, is/are simultaneously amplified.

21. The method as claimed in claim 20, wherein said one or more genes is or are overexpressed at the same time they are fermented.

22. The method as claimed claim 12, wherein, bacteria are fermented for the production of L-lysine in which one or more genes selected from the group consisting of
a) the pck gene which codes for phosphoenolpyruvate carboxykinase,
b) pgi gene which codes for glucose 6-phosphate isomerase, and
c) the poxB gene which codes for pyruvate oxidase is/are simultaneously attenuated.

23. A primer which comprises a polynucleotide sequence as claimed in claim 7 or parts thereof, and can produce DNA of genes which code for phosphatidate cytidylyl transferase by the polymerase chain reaction.

24. A hybridization probe which comprises a polynucleotide sequence as claimed in claim 7 and can isolate cDNA or genes which exhibit elevated homology with the sequence of the cdsA gene


SEQUENCE LISTING (4 sequences)

SEQ ID NO: 1 - DNA, 1300 bp, Corynebacterium glutamicum
  CDS (200)..(1090): cdsA gene
gaagtcgttgctcgcaaggaaaaggaactgatggaggtctagaagacctttatcgcaatggcttaagaattacgtcgtttcaacgtcgattgggcggggaaacgacgctttcttttgcttgcaagagtgtttggaagaattttttcgaaaatgctggcaccatcaacagtgacattgttagaaacttcaaggagaacccatgaatgaaccggagcaacatcaccggtccatgaggatgcccaaacccaaaaataatgcgggtcgagatctcaaagctgccattgctgtggggatcggactgggggtcctggttcttttggggattgtcctaagcccatggggttggtacatcctcgttgcaggttttatggctgcagcaacatgggaagttggtagcagacttaaagaaggcggctatcatttgccactgccgattatgatcatcggcggtcaggcaatcatctggctgtcatggccatttggcacgatgggcattttggcgtcttttgtggccactgtgttggtgctgatgtatttccgaattttctacaatggcacggaaaaagaagcccgcaactatttgagggacacctctgtgggcatcttcgtgctcacctggattccattgttcggaagcttcgctgcgatgctgtcgctgatgcaaaacaattccatcccgggtacatatttcattttgacgttcatgctgtgtgtgatcgcatcggatgtgggcgggtatatcgcgggtgtgttctttggatcgcacccaatggcgccgttggtgagtccgaagaagtcttgggaaggctttgccggctccattgtcttaggatcggtcactggtgcactcagtgttcacttcctgctcgatcaccactggtggatgggtgtgatcttgggttgtgccctagttgtgtgcgccacgttgggtgacttggttgagtcgcagttcaaacgcgatttgggcatcaaggatatgtcgaaccttcttccaggccacggcggattgatggaccgtttggatggcatgctcccggccgcgatggtgacgtggttgatcctgagtgtgatcagcagctcgtatccgtcgtaaagcttgggccagctttaagttcaaaaaacttgaaaggcgctgaggtgcataacgtgtgcacctcagcgcctttttggctgtcaaagtttaaagggctttacggatttttcttaactggcgacggtactcaaacatgcgcacgccaccaacaagcgccataatcaatgcaccggtaatggctgctagtaggaaaccgattccggctgg

SEQ ID NO: 2 - protein, 297 aa, Corynebacterium glutamicum
MNEPEQHHRSMRMPKPKNNAGRDLKAAIAVGIGLGVLVLLGIVLSPWGWYILVAGFMAAATWEVGSRLKEGGYHLPLPIMIIGGQAIIWLSWPFGTMGILASFVATVLVLMYFRIFYNGTEKEARNYLRDTSVGIFVLTWIPLFGSFAAMLSLMQNNSIPGTYFILTFMLCVIASDVGGYIAGVFFGSHPMAPLVSPKKSWEGFAGSIVLGSVTGALSVHFLLDHHWWMGVILGCALVVCATLGDLVESQFKRDLGIKDMSNLLPGHGGLMDRLDGMLPAAMVTWLILSVISSSYPS

SEQ ID NO: 3 - DNA, 34 bp, Artificial Sequence
  Primer for amplifying cdsA gene
cgcggatccgtggcccaagctttacgacggatac

SEQ ID NO: 4 - DNA, 33 bp, Artificial Sequence
  Primer for amplifying cdsA gene
cgcggatccggctcgcaaggaaaaggaactgat